(19) United States
(12) Patent Application Publication
(10) Pub. No.: US 2021/0322745 A1
(43) Pub. Date: Oct. 21, 2021

(54) DEVICE COMPRISING MICRONEEDLES FOR COSMETIC FILLERS DELIVERY

(71) Applicant: L'OREAL, Paris (FR)

(72) Inventors: Gaurav AGARWAL, Takatsu-ku (JP); Dominique BORDEAUX, Chevilly-La-Rue (FR); Veronique BURNIER, Chevilly-La-Rue (FR); Philippe BARBARAT, Chevilly-La-Rue (FR)

(21) Appl. No.: 17/269,131

(22) PCT Filed: Sep. 24, 2018
(86) PCT No.: PCT/EP2018/075800

§ 371 Date: Feb. 17, 2021

(51) Int. Cl. A61M 37/00; A61L 27/20; A61L 27/58

(52) U.S. Cl. CPC A61M 37/0015; A61L 27/20; A61M 2037/0061; A61M 2037/003; A61M 2037/0023; A61L 27/58

(57) ABSTRACT

A device for delivering a cosmetic filler composition to the skin including a base carrying a plurality of hollow microneedles, in which each microneedle has an external largest transverse dimension less than or equal to 1500 micrometers, and the microneedles include a filler composition.

### TECHNICAL FIELD

The present invention relates to a microneedle device comprising an array of microneedles which can be used to deliver a composition to skin, scalp, or lips to perform a cosmetic treatment, especially an anti-ageing and/or filling treatment.

### BACKGROUND ART

It is known to use injectable cosmetic fillers for diminishing facial lines and restoring volume and fullness in the face. As we age, faces naturally lose subcutaneous fat. The facial muscles are then working closer to the skin surface, so smile lines and crow's feet for example become more apparent. The facial skin also stretches a bit, adding a loss of facial volume. Other factors that affect the facial skin include sun exposure, heredity and lifestyle.

The Stratum Corneum (SC) constitutes the main barrier of the epidermis to exogenous substances, including small and high weight molecular bio-polymers compositions used as cosmetic fillers. Techniques aimed at removing the SC barrier, such as tape-stripping and suction, laser, or thermal ablation are impractical, while needle-free injections have so far failed to replace known needle-based delivery. Such a method of delivery can be uncomfortable, and even painful, due to the shape of the needles, and the viscosity of the composition, such as compositions including hyaluronic acid, and are thus non-attractive for the users.

The concept of using a microstructured device consisting of a plurality of microneedles to breach the stratum corneum barrier was first proposed in the 1970s. The production of solid microneedles arrays has been described in the art, for example in the applications WO 2009/040548, US 2015/0141910, and WO 2016/076442. Microneedles have an advantage of potentially penetrating the stratum corneum, without the discomfort of known needles, and can be self-administered.

There is a need for improving the delivery of cosmetic compositions into the skin, scalp, or lips, especially the precision of the amount of composition delivered and the control of the depth of injection into the skin at the targeted location.

### DISCLOSURE OF THE INVENTION

An object of the present invention is a device for delivering a cosmetic filler composition to the skin, comprising a base carrying a plurality of hollow microneedles, the device being characterized in that each microneedle has an external largest transverse dimension less than or equal to 1500 micrometers, and in that the microneedles comprise a filler composition.

Using the device of the invention offers a long lasting solution for skin ageing signs and disorders correction, by improving skin penetration of filler agents. When injected into skin layers, cosmetic fillers advantageously have an effect on physical, mechanical, and/or optical characteristics of the skin, inside and outside, especially on hydration, elasticity, volume suppleness, tonicity, firmness, brightness, glow, and also on the appearance of the skin surface, enhancing its softness, relief, glow, and/or color.

The device according to the invention allows targeting and delivering resorbable and injectable bio-polymers compositions into skin layers, to correct, soften and/or erase skin ageing signs disorders, such as wrinkles, skin creases, especially facial creases, skin depressions, modifications of the face oval, especially shallow contours, or the sagging of certain areas. The appearance of some recessed scars, due for example to acne, the shape and volume of cheekbones and general loss of volume may also be corrected. The invention may further be used to plump thin lips, reconstruct contour deformities of the face, decrease or remove the shadow of the lower lids.

Thanks to the hollow microneedles, the composition is delivered deeper inside skin, by-passing the SC layer. The delivery and diffusion of high molecular weight polymers is possible, even by reaching deep layers, which is not possible with topical applications.

The device of the invention is convenient to use for cosmetic, non-therapeutic, treatments.

Microneedles

The microneedles used according to the invention are known in the art.

By “hollow microneedles”, it has to be understood that the microneedles are not solid.

Hollow microneedles are disclosed in many publications such as in the articles “Microneedles for transdermal drug delivery”, Advanced Drug Delivery Reviews, Volume 56, Issue 5, 27 Mar. 2004, Pages 581-587, “Biodegradable polymer microneedles: Fabrication, mechanics and transdermal drug delivery”, Journal of Controlled Release, Volume 104, Issue 1, 5 May 2005, Pages 51-66, “Microfabricated needles for transdermal delivery of macromolecules and nanoparticles: Fabrication methods and transport studies, transport studies”, Devin V. McAllister et al., PNAS Nov. 25, 2003. 100 (24) 13755-13760, “Sharp beveled tip hollow microneedle arrays fabricated by LIGA and 3D soft lithography with polyvinyl alcohol”, F Perennes et al., published 25 Jan. 2006, IOP Publishing LtdJournal of Micromechanics and Microengineering, Volume 16, Number 3, “Microneedle array for transdermal biological fluid extraction and in situ analysis”, E. V. Mukerjeeab et al., Sensors and Actuators A: Physical, Volume 114, Issues 2-3, 1 Sep. 2004, Pages 267-275, or “Hollow Microneedle Arrays for Intradermal Drug Delivery and DNA Electroporation”, Lievin Daugimont et al., The Journal of Membrane Biology, July 2010, Volume 236, Issue 1, pp 117-125.

Hollow microneedles are also known from internet publications, such as the ones at https://www.ncbi.nlm.nih.gov/pmc/articles/PMC4588187/, concerning Transdermal Delivery of Drugs with Microneedles-Potential and Challenges, Pharmaceutics 2015 Sep.; 7(3): 90-105, published online 2015 Jun. 29, or https://www.ncbi.nlm.nih.gov/pubmed/17940907, Pharmaceutics 2015 Sep.; 7(3): 90-105, published online 2015 Jun. 29, concerning Membrane-sealed hollow microneedles and related administration schemes for transdermal drug delivery, or https://www.3m.com/3M/en_US/drug-delivery-systems-us/ . . . /microneedle/concerning 3M Microneedle Drug Delivery Systems, or https://link.springer.com/article/10.1007/s00542-012-1663-1 concerning Optimizing hollow microneedles arrays aimed at transdermal drug delivery, Microsystem Technologies, January 2013, Volume 19, Issue 1, pp 1-8, or https://www.tyndall.ie/transdermal-drug-delivery.

Hollow microneedles are also known from the patent application EP 2 876 602, describing a method for converting an image of an array of nanostructures to a representation in a coordinate system.

Patent application US 2011/0213335 discloses a rapid, high-volume, intradermal infusion with minimal pain, achieved by applying an array of several hollow microneedles into the skin of a patient.

Patent application US 2013/0116523 describes a method for fabricating a hollow microneedle having a variable appearance. The method makes it possible to vary the length of the microneedle, the outer and inner diameters of the upper and lower parts thereof, the aspect ratio, the sharpness, and the structural bending rate thereof, in accordance with the desired purposes of use.

Patent application U.S. Pat. No. 6,503,231 discloses a microneedle device for transport of therapeutic and diagnostic materials and/or energy across tissue barriers. The microneedles are hollow and/or porous and permit drug delivery, or removal or sensing of body fluids, at clinically relevant rates across skin or other tissue barriers, without damage, pain, or irritation to the tissue.

The hollow microneedles may have at least one internal channel. Such a channel may be longitudinal, that is to say running along a longitudinal axis of the microneedles, from their free ends to the base of the device, or transversal, that is to say running along an axis extending obliquely or perpendicularly to the longitudinal axis of the microneedles. The microneedles may have a plurality of channels, longitudinal and/or transversal. The channels running through the microneedles may be continuous or discontinuous.

The microneedles may have a circular hollow internal cross-section. In a variant, the microneedles have hollow internal cross-section of other shapes, for example square, rectangular, or triangular.

The microneedles may be made in a porous material, preferably able to be loaded with said at least one composition aimed at generating an in-situ reaction into the skin, scalp, or lips. The microneedles may be made in a sintered material.

The microneedles are preferably resorbable.

By “resorbable microneedles”, or “bioresorbable, or naturally-dissolving”, it has to be understood that the microneedles dissolve or degrade in vivo, for example after between at least 10 seconds and 24 hours, preferably in less than 8 hours. The microneedles may be bioabsorbable or biodegradable. The microneedles may dissolve or degrade thanks to an enzymatic reaction. In the case of resorbable microneedles, the microneedles are advantageously manufactured from a material that may dissolve, be absorbed or broken down by the body and thus do not require any mechanical removal.

The microneedles may be soluble in any body fluid or suitable composition.

In a variant, resorbable microneedles may be swellable into the skin, scalp or lips, that is to say being able to increase in volume when injected into the skin, scalp or lips.

In the case of resorbable microneedles, at least part of the material in which are made the microneedles may be used, once injected into the skin, scalp or lips, as a cosmetic filler.

In a variant, the microneedles are non-resorbable.

By “non-resorbable microneedles”, it has to be understood that the microneedles do not dissolve or degrade in vivo, and that they have to be removed from the skin. The microneedles may be not moisture-swellable.

A length of each microneedle is preferably less than or equal to 50 mm, better to 20 mm, better to 5 mm, better to 1 mm, better to 0.3 mm, even better to 0.1 mm. The length of a microneedle is measured, along its elongation axis, from its free end to the point where it connects to the base. The expression “elongation axis of a microneedle” denotes an axis which passes through the centers of mass of the cross sections of the microneedle.

The external largest transverse dimension of each microneedle, measured at the point where it is attached to the base, perpendicularly to its elongation axis, may be less than or equal to 1000 micrometers, better to 300 micrometers. The external cross-dimension of the microneedles advantageously decreases regularly towards their free ends.

The internal volume of each microneedle may be less than or equal to 10 mm³, better than 5 mm³, even better than 3 mm³.

The microneedles may be configured to deliver the composition at a flow rate less than or equal to 3 cm³/min, better to 0.3 cm³/min.

Each microneedle may comprise a stop configured for limiting the depth of injection of the microneedle into the skin to less than or equal to 500 micrometers, better to 200 micrometers, even better to 100 micrometers.

An internal largest transverse dimension of each microneedle, i.e. its diameter when the microneedle has a circular hollow cross-section, may be less than or equal to 1000 micrometers, better to 500 micrometers, even better to 200 micrometers. The internal largest transverse dimension of the microneedles may be chosen according to the required volume of composition to be distributed.

The microneedles are preferably longer than the desired depth of injection. The length of the microneedles may be chosen according to the targeted skin layer into which the composition has to be delivered, obtaining the appropriated depth into the skin.

The microneedles may be oriented perpendicular to a substantially planar surface along which the base extends. In a variant embodiment, the microneedles are oriented obliquely to the substantially planar surface along which the base extends. This allows delivering the composition to curved surfaces with locally oriented perpendicularly microneedles.

The microneedles may have a curved shape.

The microneedles may be of non-cylindrical shape, especially pyramidal with an octagonal base. The article of O'Mahony C., “Structural characterization and in-vivo reliability evaluation of silicon microneedles”, Biomed Microdevices, 2014, 16(3):333-43 shows that conical shape leads to very high reliability for silicon microneedles during skin penetration. In variants, the microneedles may be of conical shape with other polygonal bases such as a hexagonal base, or of square shape.

The microneedles may be made of an inorganic material, preferably silicon, titanium, stainless steel, cobalt, ceramics, polyethylene, or any material than can be skin and/or body implantable. The material used for the microneedles may include a preservative, especially in the case of ceramics.

In a variant, the microneedles are made of an organic material, preferably a polymer, for example a Gantrez polymer, or sugar, polysaccharide, polyethylene, cellulose, or hyaluronic acid.

The material used for the microneedles may be able to carry light and/or heat and/or cold.

The microneedles are preferably sterile or sterilized before use. The microneedles are preferably of single use.

Device, Control, Communication

The base may be a flexible substrate, preferably coated with adhesive.

The device may comprise a plurality of rigid bases each carrying one or more microneedles, preferably integral therewith, and a flexible support to which the bases are fixed so that the bases can move relative to the other to conform to skin profile.

The device may be made of several bases connected together to one or different containers containing a composition to be injected.

The containers may be removed from the device and replace by a new one while the microneedles are still into the skin.

The containers may be refillable while still connected to an array of microneedles.

The base may carry between 2 and 1000 microneedles per cm², better between 15 and 50 microneedles. The number of microneedles by base advantageously depends on the targeted area of the skin to be treated. The number of microneedles on a base is advantageously related to the size of the base, which depends on the size of the area of the skin to be treated.

The device may be able to make the microneedles puncturing the skin at a deep level, then to distribute the composition while removing the device. This allows having a good distribution of the composition in the different skin layers.

The microneedles may be disposed regularly on the base. In a variant, the microneedles are non-regularly disposed on the base.

The base may carry only resorbable microneedles, or only non-resorbable microneedles. In a variant, the base carries a mix of resorbable and non-resorbable microneedles. The ratio of resorbable microneedles and non-resorbable microneedles may be between 0 and 1.

The microneedles of a base may have different lengths, different volumes, different shapes, and may be made of different materials.

The device may be connected by wireless communication to an electronic system, in particular a personal computer or a smartphone, for injection control. In a variant, the device comprises an electronic system.

Such electronic system may be arranged for computing for a set of injection points of said area, obtained from an optical acquisition of the topography of the area, a volume of filler to introduce into the skin to obtain a desired correction of the relief of this area, and computing a volume of filler to inject into the skin, scalp, or lips via each microneedle based on the volume computed in each point and localization of each microneedle on said area during the injection of filler. This allows obtaining great precision and avoiding errors, especially by preventing the risks of excess of composition and of overflow beyond the area to be filled.

The device may be able to deliver heat, cold, ultrasounds, massage, microcurrents, or light, to create a synergic effect on composition performance or to enable chemical reactions of the different compositions. Moreover, heat can be useful to maintain a low viscosity of the composition to ease its injection. Cold may help to harden the skin and to help skin being punctured by the microneedles. This delivering may be controlled by the electronic system.

The targeted area of skin may be sucked up into a chamber to bring the targeted area into contact with the microneedles. Sucking the skin helps the microneedles puncturing the skin, and also having the same penetration depth of all the microneedles into the skin.

To this end, it may be used a sucking device comprising:

a chamber with an opening configured to face the targeted area of the skin when the device is applied on the skin,

a surface within the chamber at a recessed position from the opening, and

a pressure source in communication with the chamber, at least for decreasing the pressure in the chamber and causing the targeted area to be sucked up in the chamber and to contact the surface,

the surface comprising the microneedles according to the invention for puncturing the skin of the targeted area.

An application, in particular running on a smartphone, may be used for delivery control and user interface.

The device may be part of a watch connected to a smartphone, or to a distant device connected with a wire or wireless, for example by radio frequency, WIFI, or Bluetooth®.

A gas may be injected by the microneedles, before or after the delivery of the filler, preferably a carbon dioxide (CO₂). Such a gas injection is known to allow increasing blood flow into the skin, which permits to increase compounds exchanges between compositions and the skin and also to temporary create skin bumps beneficial for wrinkle size diminution. Carbon dioxide may be created while mixing a calcium carbonate solution (CaCO³) with an acid, as citric acid. Both components are compatible for injection into the skin. Other minerals and acids may be mixed to generate gas formation into the skin.

The device may comprise a vibrating system for inducing vibrations into the microneedles. Such a vibrating system may allow the microneedles to puncture the skin, avoiding fluid blockage in the microneedles, and then ease removing non-resorbable microneedles from the skin after delivery of the composition. The vibrating system may further provide a massage to the treated area, allowing a better diffusion of the composition.

System and Compositions

Another object of the invention is a system comprising the device as defined above and at least one cosmetic filler composition to be injected.

The composition may be of same rheology before and after delivery into the skin.

The composition is preferably a biocompatible cosmetic composition. By “biocompatible”, it has to be understood a composition capable of fulfilling a specific function with an appropriate response from the skin.

The composition is preferably sterile, and of single use.

The cosmetic filler preferably comprises at least one bio-polymer. Thanks to bio-polymers mechanism and the injection at high quantity and at the right depth into sites which cannot be reached using topical applications, long lasting and visible effects are obtained.

The cosmetic filler may be resorbable, that is to say may completely dissolve into the skin, in 3 to 6 months, or in 6 to 24 months, which corresponds to slowly resorbable. In a variant, the composition is definitive, non-resorbable.

On the long term, thanks to the invention, the own collagen production of the user may be stimulated thanks to the delivered filler, and long lasting effects of filling can be observed

Any kind of injectable bio-polymer acceptable for skin injection purposes may be used, as for example the ones allowed by the American Food and Drug Administration (FDA) or the French “Agence nationale de sécurité du médicament et des produits de santé” (ANSM).

The viscosity of the composition may range from 1.10⁻³ Pa s⁻¹ to 10000 Pa s⁻¹, preferably from 1.10⁻³ Pa s⁻¹ to 3000 Pa s⁻¹.

The viscosity is measured at 25° C. and under 1 atm, with a Rheomat 180 viscometer equipped with MK-R-1, 2 or 3 mobile according to the viscosity range and the corresponding measurement cup MB-R-1, 2 or 3 at a rotation speed of 200 min⁻¹, the measurement being carried out after 10 minutes of rotation (time at which the stabilization of the viscosity and the speed of rotation of the mobile is observed).

The cosmetic filler may comprise at least one of hyaluronic acid from low to high molecular weight, and/or crosslinked, hydroxyapatite particles, polylactic acid, alginate, monomethyltrisilanol orthohydroxy benzoate de sodium, polyacrylamide, a mix of hyaluronic acid and lidocaine, or a mix of hyaluronic acid and dextran microbubbles, and any mixture of the above compounds and mixes.

The cosmetic filler may be associated with at least one active compound or molecule or polymer to get added benefits.

The cosmetic fillers considered according to the invention are in particular chosen from (R: resorbable in 3 to 6 months, LR=SR: slowly resorbable in 6 to 24 months, NR: non-resorbable, definitive):

| | | | Resorbable |
|---|---|---|---|
| Commercial Name | Fabricant | Composition | or not |
| Achyal ® | TEDEC-MEIJI | Hyaluronic Acid | R |
| | FARMA SA | | |
| Hyaluronic Acid | Vital Esthétique | Hyaluronic Acid | R |
| HYALURONICA-1- ® | | | |
| Hyaluronic Acid | Vital Esthétique | Hyaluronic Acid | R |
| HYALURONICA-2- ® | | | |
| Dermyal 18 ® | Hyal Intertrade | Hyaluronic Acid | R |
| Dermyal 24r ® | Hyal Intertrade | Hyaluronic Acid | R |
| Dermyal 32 hr ® | Hyal Intertrade | Hyaluronic Acid | R |
| Esthelis Soft ® | Anteis Sa | Hyaluronic Acid | R |
| Esthelis Basic ® | Anteis Sa | Hyaluronic Acid | R |
| Fortelis Extra ® | Anteis Sa | Hyaluronic Acid | R |
| GLYTONE Professional | Pierre Fabre Dermo- | Hyaluronic Acid | R |
| 2 ® Rides Superficielles | Cosmetique | | |
| GLYTONE Professional | Pierre Fabre Dermo- | Hyaluronic Acid | R |
| 3 ® Rides Moyennes \| | Cosmetique | | |
| Profondes | | | |
| Hyaluderm 2% ® | Lca Pharmaceutical | Hyaluronic Acid | R |
| Hyaluderm 2.5% ® | Lca Pharmaceutical | Hyaluronic Acid | R |
| Idune ® 2.0% | Genevrier | Hyaluronic Acid | R |
| Idune ® 1.6% | Genevrier | Hyaluronic Acid | R |
| Idune ® 0.8% | Genevrier | Hyaluronic Acid | R |
| ISOGEL ® Class I, II, III | Laboratoires FILORGA | Hyaluronic Acid | R |
| Jolidermis 24 ® | Anteis Sa | Hyaluronic Acid | R |
| Joless Define ® | Hyaltech Limited | Hyaluronic Acid | R |
| Joless Balance ® | Hyaltech Limited | Hyaluronic Acid | R |
| Joless Soft ® | Hyaltech Limited | Hyaluronic Acid | R |
| Jolidermis 24+ ® | Anteis Sa | Hyaluronic Acid | R |
| Jolidermis 18 ® | Anteis Sa | Hyaluronic Acid | R |
| Lissant-Yal 1.6% ® | Laboratoires Pharmy 2 | Hyaluronic Acid | R |
| Lissant-Yal 2.2% ® | Laboratoires Pharmy 2 | Hyaluronic Acid | R |
| M-Ha18 ® | Filorga | Hyaluronic Acid | R |
| Perfectha Derm ®-Pd | Obvieline | Hyaluronic Acid | R |
| Perfectha Derm ® Deep- | Obvieline | Hyaluronic Acid | R |
| Pdd | | | |
| Perfectha Derm ® Fine | Obvieline | Hyaluronic Acid | R |
| Lines-Pdfl | | | |
| Prevelle ® | Genzyme Biosurgery | Hyaluronic Acid | R |
| Prevelle ® silk | Genzyme Biosurgery | Hyaluronic Acid with | R |
| | | lidocaine | |
| Princess ® Filler | Croma Gmbh | Hyaluronic Acid | R |
| Princess ® Rich | Croma Gmbh | Hyaluronic Acid | R |
| Puragen ® | Mentor Biopolymers | Hyaluronic Acid | R |
| | Ltd | | |
| Renofill Deeply | Laboratoires | Hyaluronic Acid | R |
| Correction ® | Renophase | | |
| Renofill Perfectly | Laboratoires | Hyaluronic Acid | R |
| Volume ® | Renophase | | |
| Renofill Softy | Laboratoires | Hyaluronic Acid | R |
| Correction ® | Renophase | | |
| Revanesse ® | Prollenium Medical | Hyaluronic Acid | R |
| | Technologies Inc | | |
| Revanesse ® Lips ® | Prollenium Medical | Hyaluronic Acid | R |
| | Technologies Inc | | |
| Revanesse ® Pure | Prollenium Medical | Hyaluronic Acid | R |
| | Technologies Inc | | |
| Revanesse ® Ultra | Prollenium Medical | Hyaluronic Acid | R |
| | Technologies Inc | | |
| R-Fine ® | Hyaltech Limited | Hyaluronic Acid | R |
| PRINCESS VOLUME ® | CROMA Gmbh | Hyaluronic Acid | R |
| Stylage ® Xxl | Laboratoires Vivacy | Hyaluronic Acid | R |
| STYLAGE Hydromax ® | Laboratoires VIVACY | Hyaluronic Acid | R |
| STYLAGE S 0.4 ml ® | Laboratoires VIVACY | Hyaluronic Acid | R |
| Stylage Xl ® | Laboratoires Vivacy | Hyaluronic Acid | R |
| Stylage ® L | Laboratoires Vivacy | Hyaluronic Acid | R |
| Stylage ® Hydro | Laboratoires Vivacy | Hyaluronic Acid | R |
| Stylage ® M | Laboratoires Vivacy | Hyaluronic Acid | R |
| Stylage ® M Lidocaine | Laboratoires Vivacy | Hyaluronic Acid | R |
| Stylage ® S | Laboratoires Vivacy | Hyaluronic Acid | R |
| Stylage ® S Lidocaine | Laboratoires Vivacy | Hyaluronic Acid | R |
| Stylage ® Special Lips | Laboratoires Vivacy | Hyaluronic Acid | R |
| Stylage ® Spécial Lips | Laboratoires Vivacy | Hyaluronic Acid | R |
| Lidocaine | | | |
| Succeev One ® | Hyal Intertrade | Hyaluronic Acid | R |
| Succeev Two ® | Hyal Intertrade | Hyaluronic Acid | R |
| Succeev Three ® | Hyal Intertrade | Hyaluronic Acid | R |
| Surgiderm 18 ® | Allergan | Hyaluronic Acid | R |
| Surgilift Plus ® | Allergan | Hyaluronic Acid | R |
| Varioderm ® Mesolift | Adoderm Gmbh | Hyaluronic Acid | R |
| Visagel ® | Surgical Concepts | Hyaluronic Acid | R |
| | Gmbh | | |
| X-Ha3 ® | Laboratoires Filorga | Hyaluronic Acid | R |
| X-HA Volume ® | Laboratoires FILORGA | Hyaluronic Acid | R |
| Conjonctyl ® | Sedifa Laboratoires | Monomethyltrisilanol | LR |
| | | Orthohydroxy Benzoate | |
| | | Sodium | |
| Hydrafill Soft Line ® | Allergan | Hyaluronic Acid | LR |
| Juvederm Ultra 2 ® | Allergan | Hyaluronic Acid | LR |
| Juvederm Ultra 3 ® | Allergan | Hyaluronic Acid | LR |
| Juvederm Ultra 4 ® | Allergan | Hyaluronic Acid | LR |
| Juvederm Ultra Lip ® | Allergan | Hyaluronic Acid | LR |
| Juvederm ® Ultra Smile ® | Allergan | Hyaluronic Acid | LR |
| Juvederm ® Voluma, | Allergan | Hyaluronic Acid | LR |
| Voluma XC | | | |
| Juvederm ® 24HV, 30, 30 | Allergan | Hyaluronic Acid | LR |
| HV | | | |
| Juvederm ® Ultra XC, | Allergan | Hyaluronic Acid with | LR |
| Ultra Plus XC | | lidocaine | |
| Juvederm ® Volbella XC | Allergan | Hyaluronic Acid with | LR |
| | | lidocaine | |
| Juvederm ® Vollure XC | Allergan | Hyaluronic Acid | LR |
| New-Fill ® | Dermik Laboratories (A | Polylactic acid | LR |
| | Business Of Sanofi- | | |
| | Aventis Us Llc) | | |
| Radiesse ® | Merz Aesthetics Inc | Hydroxyapatite De Calcium | LR |
| Radiesse ® 1.3 CC, 0.3 CC | Bioform Medical, Inc | Hydroxyapatite De Calcium | LR |
| Restylane Lidocaine ® | Q-Med Ab | Hyaluronic Acid | LR |
| Restylane Perlane | Q-Med Ab | Hyaluronic Acid | LR |
| Lidocaine ® | | | |
| Restylane Perlane ® | Q-Med Ab | Hyaluronic Acid | LR |
| Restylane Subq ® | Q-MED AB | Hyaluronic Acid | LR |
| Restylane Touch ® | Q-Med Ab | Hyaluronic Acid | LR |
| Restylane Vital “Pen | Q-MED AB | Hyaluronic Acid | LR |
| Injector” (Stylo | | | |
| D'injection) ® | | | |
| Restylane Vital Light ® | Q-Med Ab | Hyaluronic Acid | LR |
| Restylane Vital Light- | Q-Med Ab | Hyaluronic Acid | LR |
| Injector ® | | | |
| Restylane Vital White ® | Q-Med Ab | Hyaluronic Acid | LR |
| Restylane Vital White- | Q-Med Ab | Hyaluronic Acid | LR |
| Injector ® | | | |
| Restylane Vital ® | Q-Med Ab | Hyaluronic Acid | LR |
| Restylane ® Refyne, | Q-Med Ab | Hyaluronic Acid | LR |
| Defyne | | | |
| Restylane ® L injectable | Medicis Aesthetics | Hyaluronic Acid with | LR |
| gel | Holdings, Inc | lidocaine | |
| Restylane ® Silk | Q-MED AB | Hyaluronic Acid with | LR |
| | | lidocaine | |
| Restylane ® Lyft with | Q-MED AB | Hyaluronic Acid with | LR |
| lidocaine | | lidocaine | |
| Restylane ® | Q-MED AB | Hyaluronic Acid | LR |
| Sculptra ® | Dermik Laboratories (A | Polylactic acid | LR |
| | Business Of Sanofi- | | |
| | Aventis Us Llc) | | |
| Sculptra ® Aesthetic | Sanofi Aventis US | Polylactic acid | LR |
| Surgiderm 24xp ® | Allergan | Hyaluronic Acid | LR |
| Surgiderm 30 ® | Allergan | Hyaluronic Acid | LR |
| Surgiderm 30xp ® | Allergan | Hyaluronic Acid | LR |
| Surgilips ® | Allergan | Hyaluronic Acid | LR |
| Teosyal 27 g Deep Lines | Teoxane Sa | Hyaluronic Acid | R |
| Single ® | | | |
| Teosyal 27 g Deep Lines ® | Teoxane Sa | Hyaluronic Acid | R |
| TEOSYAL 30 G Global | TEOXANE SA | Hyaluronic Acid | R |
| Action (2 × 1 ml) ® | | | |
| Teosyal First Lines ® | Teoxane Sa | Hyaluronic Acid | LR |
| TEOSYAL 30 G Global | TEOXANE SA | Hyaluronic Acid | R |
| Action Single (1 ml) ® | | | |
| Teosyal Puresense First | TEOXANE SA | Hyaluronic Acid + Lidocaine | LR |
| Lines ® | | | |
| Teosyal Puresense Global | TEOXANE SA | Hyaluronic Acid + Lidocaine | LR |
| Action ® | | | |
| Teosyal Puresense Kiss ® | TEOXANE SA | Hyaluronic Acid + Lidocaine | LR |
| Teosyal Puresense | TEOXANE SA | Hyaluronic Acid + Lidocaine | LR |
| Ultradeep ® | | | |
| Teosyal Puresense | TEOXANE SA | Hyaluronic Acid + Lidocaine | LR |
| Deeplines ® | | | |
| Teosyal 30 g Touch Up ® | Teoxane Sa | Hyaluronic Acid | R |
| Teosyal Kiss Single ® | Teoxane Sa | Hyaluronic Acid | R |
| Teosyal Kiss ® | Teoxane Sa | Hyaluronic Acid | R |
| Teosyal Ultra Deep | Teoxane Sa | Hyaluronic Acid | R |
| Single ® | | | |
| Teosyal Ultra Deep ® | Teoxane Sa | Hyaluronic Acid | R |
| Varioderm ® | Adoderm Gmbh | Hyaluronic Acid | LR |
| VARIODERM ® Plus | ADODERM Gmbh | Hyaluronic Acid | LR |
| VARIODERM ® | ADODERM Gmbh | Hyaluronic Acid | LR |
| Lips & Medium | | | |
| Varioderm ® Fine Line | Adoderm Gmbh | Hyaluronic Acid | LR |
| X-HA Volume ® | FILORGA | Hyaluronic Acid | LR |
| Redexis ® | Prollenium Medical | Microball Dextran + | NR |
| | Technologies Inc | Hyaluronic Acid | |
| Aquamid ® | Ferrosan A/S | Polyacrylamid | NR |
| Novabel ® | Merz Pharmaceuticals | Alginate | LR |
| | Gmbh | | |
| Biostyle ® | Sun | Hyaluronic Acid | LR |
| | COMMUNICATION | | |
| Hyalskin ® | Mesoface Choc Medical | Hyaluronic Acid | LR |
| Reparestim Ha ® | | | |
| Zetaderm ®, Zetavisc ®, | Philoderm Aesthetics | Hyaluronic Acid | LR |
| Philoderm ® Professional | | | |
| Mesoface ® | | | |
| Amalian ® | S&V Technologies Ag | Hyaluronic Acid | R |
| JOLIESSE ®, JOLIESSE | France LENS | Hyaluronic Acid | R |
| Plus ® | | | |
| Evolence ® collagen filler | Colbar Lifscience | Collagen | LR |
| Outline ® Fine/Outline | Procytech | Modified Polyacrylamid | LR |
| Original/Outline Ultra | | | |
| Evolution ® | Procytech | Polyacrylamid | NR |
| Novasoft ® | Adoderm Gmbh | Hyaluronic Acid + Particles | NR |
| | | of Polymers | |
| | | (Poly(Methacrylate | |
| | | hydroxyethyle-Co- | |
| | | Methacrylate Ethyl) (Phema- | |
| | | Co-Ema) | |
| Zyplast ® | Collagen Corp. | Collagen | |
| Zyderm ® Collagen | Collagen Allergan | Collagen | |
| Implant | | | |
| Fibrel ® | Serono Laboratories | Collagen | |
| Cosmoderm ® 1 human- | Inamed Corporation | Collagen | |
| based C | | | |
| Hylaform ® (Hylan B gel) | Genzyme Biosurgery | Modified hyaluronic acid | |
| Captique ® injectable gel | Genzyme Biosurgery | Hyaluronic Acid | |
| Artefill ® | Suneva Medical, Inc | Polymethylmethacrylate | |
| | | Beads, Collagen and | |
| | | Lidocaine | |
| Elevess ® | Anika Therapeutics | Hyaluronic Acid with | |
| | | lidocaine | |
| Belotero Balance ® | Merz Pharmaceuticals | Hyaluronic Acid | |
| Dermalive/Dermadeep ® | Dermatech | Hyaluronic Acid + Particles | NR |
| | | of Polymers | |
| | | (Poly(Methacrylate | |
| | | hydroxyethyle-Co- | |
| | | Methacrylate ethyle) (Phema- | |
| | | Co-Ema) | |

All the providers of these fillers can be found on the internet.

The features defined above for the device apply to the system and vice-versa.

Flexible System

The device according to the invention may be embedded on flexible and wearable system arranged for conforming to the area of the skin where the composition has to be delivered.

Such a wearable system may be made of a non-woven, absorbent material such as for example foam, latex, polyurethane, or film. Such a wearable system may be made of a resorbable material.

The thickness of the wearable system may lie between 5 μm to 3 mm, preferably between 15 μm to 500 μm.

The wearable system may include a holding device to help its fixing on the treated area of the skin. The wearable system may include an adhesive polymer for fixing.

Kit

Yet another object of the invention is a kit comprising the device according to the invention and one or several cosmetic fillers.

The cosmetic fillers may be as defined above.

The features defined above for the device apply to the kit and vice-versa.

Method for Preparing an Injection of Cosmetic Filler

Yet another object of the invention is a method for preparing an injection of a cosmetic filler into an area of skin, scalp, or lips for correcting a relief of this area, the injection being performed with the device as defined above, the method comprising:

performing an acquisition of the topography of the area, preferably an optical acquisition, and

based on such acquisition of the topography, computing a volume of filler to inject into the skin, scalp, or lips via each microneedle.

Such computing may take into account the localization of the microneedles on said area during the injection of filler when the device allows for injection of different quantities of filler between at least two microneedles. For example, more filler is injected into the skin via some microneedles where the skin needs locally more filler to fill a hollow. The quantity of filler is thus adjusted very precisely depending on the local need for filler to achieve the desired relief correction.

The computing of the volume of composition to inject may also depend on the depth of injection and on the localization of the microneedles on the area to treat when successive injections are carried out with displacement of the device relative to the area to be treated from one injection to the other. For example, the device will inject a first quantity of filler when in a first position, then be removed and placed in a second position next to the first one, and then a second quantity of filler is injected, that is different from the first one, because for instance the need of filler locally is less. Accordingly, the device may compute a succession of volumes of filler to inject for respective injection sites. In such a case, each microneedle of the device may inject the same quantity of filler when injection takes place in one site, but this quantity varies when the device is positioned in another site.

The internal largest transverse dimension of the microneedles may vary to enable different volume distribution in different sites, and also to help more viscous or less viscous composition to go through the microneedles. This allows a fine tuning of the composition delivery.

The position of the device on the skin, scalp, or lips may be automatically detected. The quantity of filler to be locally injected may be controlled as a function of the detected position and of the correction to be applied.

Thanks to the invention, the amount of delivered composition is finely adjusted.

The acquisition of the topography of the area may be performed by a 3D-scan or a profile-meter.

Methods to assess inskin modifications may be used, such as Optical Coherence Tomography (OCT), confocal microscopy, quantitative and qualitative assessment methods. OCT method may be used to monitor and locate injection sites into the skin, by using for example fluorescent molecules, radio tags, specific dosages. OCT method can be useful to follow the volume filled inside the skin, scalp, or lips. Confocal microscopy can be useful to follow fluorescent molecules or polymers. The injected volume may be controlled thanks to a scale or a volume measurement device. An OCT portable device may be used.

It is also possible to follow the evolution of the injected solution in skin explants by freezing them after injection and performing a mechanical sagittal cut. Bioreactions can be assessed by biomarkers analysis.

In a preferred embodiment, the microneedles are pre-filled with the composition before the application of the base on the skin. This allows avoiding the risk of injection of air.

In a variant, the microneedles are empty when the support is applied on the skin, and filled with the composition after the application on the skin.

Method of Cosmetic Treatment

Another object of the invention is a method of cosmetic treatment for correcting a relief of an area of skin, comprising delivering into the area a filler composition prepared by the method as defined above.

The depth of injection of the microneedles into the skin is preferably less than or equal to 500 micrometers, better to 200 micrometers, even better to 100 micrometers. This range of depths corresponds to the area above the dermo-epidermal junction, and avoids bleeding and thus offers a comfortable use of the device.

The depth of injection into the skin advantageously depends on the composition: lighter, finer compositions can be associated with superficial injection whilst heavier, coarser compositions are preferably injected deeper.

A pre-solution may be applied beforehand on the skin to accelerate solubility kinetics of the microneedles and allow their removal, if needed, after delivery of the composition, especially a solution, or any composition compatible for injection into the skin and/or topical application, for example a saline solution. Such a pre-solution may help to prepare skin but also to ease skin perforation. Such a pre-solution may contain active compounds and or polymers dedicated for skin surface treatment and or diffusion.

As explained above, the injection may take place with different quantities of filler depending on the localization of the microneedles on the skin, scalp, or lips. The features defined above for the device apply to the methods and vice-versa.

### DESCRIPTION OF THE DRAWINGS

The foregoing aspects and many of the attendant advantages of the disclosed subject matter will become more readily appreciated as the same become better understood by reference to the following detailed description, when taken in conjunction with the accompanying drawings, wherein:

FIG. 1 shows a schematic embodiment of a microneedle device according to the invention; and

FIG. 2 is a block diagram illustrating some steps of a method for preparing an injection of a cosmetic filler into an area of skin, scalp, or lips, according to the invention.

### DETAILED DESCRIPTION

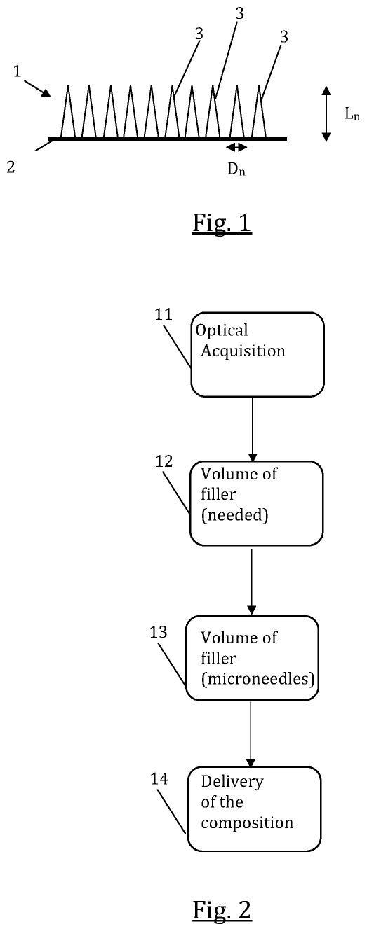

A device 1 according to the invention for delivering a cosmetic filler composition to the skin is shown in FIG. 1. In this example, such a device 1 comprises a base 2 carrying a plurality of hollow and non-resorbable microneedles 3, each microneedle having an internal channel 3a running from their free ends to the base 2, presenting an internal volume for containing the composition to deliver to the skin up to 10 mm³.

The microneedles 3 are advantageously configured to deliver the composition at a flow rate less than or equal to 3 cm³/min.

The base 2 is for example a flexible substrate.

The length Lₙ of each microneedle 3 is less than or equal to 50 mm, being for example equal to 0.7 mm. The microneedle length is advantageously adjusted to target beyond the SC barrier and/or epidermis.

The external largest transverse dimension D, of a microneedle 3, visible in FIG. 1, is for example less than or equal to 1500 micrometers.

Preferably, the microneedles are of pyramidal shape with an octagonal base, and are oriented perpendicular to a substantially planar surface along which the base extends.

The microneedles are made of an inorganic material, for example silicon, or of an organic material, for example a polymer.

The device according to the invention may comprise a vibrating system, not shown, for inducing vibrations into the microneedles 3. These vibrations may at least allow removing the non-resorbable microneedles from the skin after delivery of the composition.

In a variant, a pre-solution is applied beforehand on the skin to weaken the microneedles 3 and allow their removal after delivery of the composition, especially a saline solution.

Preferably and as in the considered example, the filler comprises at least one bio-polymer, for example at least one of hyaluronic acid from low to high molecular weight, and/or crosslinked, hydroxyapatite particles, polylactic acid alginate, monomethyltrisilanol orthohydroxy benzoate de sodium, polyacrylamide, lidocaine, dextran microbubbles, and any mixture of the above compounds and mixes.

Some exemplary steps of a method, according to the invention, for preparing an injection of cosmetic filler P into an area of skin, scalp, or lips for correcting a relief of this area, for example wrinkles, skin creases, skin depressions, or modifications of the face oval, using the microneedles device 1, are now going to be described, in reference with FIG. 2.

In a step 11, an optical acquisition of the topography of the area is performed, for example by using a 3D-scan.

For a set of injection points of said area, in a step 12, a volume of filler P to introduce into the skin to obtain a desired correction of the relief of this area is computed. Such computation may use a model giving skin relief modification as a function of the quantity injected. Such a model advantageously uses mechanobiology knowledge.

In a step 13, a volume of filler to inject into the skin, scalp, or lips via each microneedle 3 is computed, based on the volume computed in each point and on the localization of each microneedle on said area during the injection of filler. This allows for example to take into account the distance between a point where a volume to be injected has been computed and the one or more microneedles closest to this point when the injection takes place.

It is also possible to compute the quantity of filler to inject in each point and to position exactly the microneedles in these points.

The filler composition is then delivered into the area for correcting its relief, in a step 14. In this example, the microneedles 3 are pre-filled with the composition before application of the base 2 on the area of the skin. The depth of injection of the microneedles 3 into the skin is preferably less than or equal to 500 micrometers.

As previously defined, the device 1 may be connected by wireless communication to an electronic system, not shown, in particular a personal computer or a smartphone, for injection control, or comprises an electronic system. Such electronic system is in particular arranged for controlling the injection of a predefined quantity of filler P into the microneedles 3, by performing at least some of the previously-described method steps. An application, in particular running on a smartphone, may be used for delivery control and user interface.

The device 1 may comprise one or several wearable batteries, not shown, for electric power supply of the device.

### Example 1

In a first example, cross-linked hyaluronic acid on a gel form is delivered into an area of the skin according to the invention and by using a microneedles device 1. Restylane filler is used for filling fine lines of the epidermis, and a wrinkle, while Restylane Perlane filler is used for filling a skin fold. These zones are filled thanks to the invention. The injection of the filler has been prepared as previously described: by computing a volume of filler to inject into the skin, scalp, or lips via each microneedle 3, based on a computed volume of filler P to introduce in each point of the area and on the localization of each microneedle on said area during the injection of filler.

The container may comprise several rooms; each room being able to contain a different composition. The different rooms may be connected together. The container may be a syringe.

### Example 2

In a second example, Calcium Hydroxyapatite microspheres are used to fill an area where wrinkles have developed, inducing a loss of volume and shape. The filler is delivered to the skin according to the invention, and the gel increases volume immediately. On the long term, the own collagen production of the user is stimulated thanks to the Calcium Hydroxyapatite filler, and long lasting effects of filling can be observed.

Histology slides can show the deposition of new collagen around Calcium Hydroxyapatite microspheres over an extended period of time: collagen fibers stain dark, while other tissue elements appear lighter. Thanks to the invention, the production of collagen is increased after several months, even after a year.

### Example 3

In a third example, Polylactic Acid filler is delivered, by using a microneedles device 1, to an area of the skin where a skin depression has developed. By a mechanical effect, the injected volume immediately fills the skin depression. In a few days, the skin depression lightly appears again after the absorption of the water contained in the filler. The connective tissues are then restructured and the skin depression is reduced. The skin depression is visibly reduced due to conjunctive tissues proliferation.

The table below shows examples of areas of the face where some fillers can be applied thanks to microneedles array according to the invention.

| | Area | Filler |
|---|---|---|
| 1 | Forehead | Belotero Balance |
| 2 | Glabella | Restylane, Juvederm, |
| | | Belotero Balance |
| 3 | Temples | Radiesse, Juvederm, Perlane |
| 4 | Lower eyelid hollowing and | Restylane, Juvederm Ultra, |
| | tear trough | Belotero Balance |
| 5 | Nose | Radiesse, Juvederm, |
| | | Restylane |
| 6 | Cheek, midface hollowing | Radiesse, Juvederm, Perlane |
| 7 | Nasolabial fold | Radiesse, Juvederm, |
| | | Restylane |
| 8 | Fine lines | Restylane, Belotero Balance |
| 9 | Lips | Juvederm, Restylane |
| 10 | Chin | Radiesse, Juvederm, |
| | | Restylane |
| 11 | Prejowl sulcus | Radiesse, Juvederm, |
| | | Restylane |
| 12 | Ear lobes | Juvederm, Restylane |

While illustrative embodiments have been illustrated and described, it will be appreciated that various changes can be made therein without departing from the spirit and scope of the claimed subject matter.

1. A device for delivering a cosmetic filler composition to the skin, comprising a base carrying a plurality of hollow microneedles, the device being characterized in that each microneedle has an external largest transverse dimension less than or equal to 1500 micrometers, and in that the microneedles comprise a filler composition.

2. The device of claim 1, wherein the microneedles are resorbable.

3. The device of claim 2, wherein the microneedles are swellable.

4. The device of claim 1, wherein the microneedles are non-resorbable.

5. The device of claim 1, wherein the microneedles are made in a porous material.

6. The device of claim 1, wherein a length of each microneedle is less than or equal to 50 mm.

7. The device of claim 1, wherein the internal volume of each microneedle is less than or equal to 10 mm³.

8. The device of claim 1, wherein the microneedles are configured to deliver the composition at a flow rate less than or equal to 3 cm³/min.

9. The device of claim 1, wherein each microneedle comprises a stop configured for limiting the depth of injection of the microneedle into the skin to less than or equal to 500 micrometers.

10. The device of claim 1, wherein the external largest transverse dimension each microneedle is less than or equal to 1000 micrometers.

11. The device of claim 1, wherein an internal largest transverse dimension of each microneedle is less than or equal to 1000 micrometers.

12. A system comprising the device according to claim 1 and at least one cosmetic filler composition to be injected.

13. The system of claim 12, wherein the filler comprises at least one bio-polymer.

14. The system of claim 12, wherein the filler comprises at least one of hyaluronic acid from low to high molecular weight, and/or crosslinked, hydroxyapatite particles, polylactic acid alginate, monomethyltrisilanol orthohydroxy benzoate de sodium, polyacrylamide, lidocaine, dextran microbubbles and mixtures thereof.

15. A kit comprising a device such as defined in claim 1 and one or several cosmetic fillers.

16. A method for preparing an injection of a cosmetic filler into an area of skin, scalp, or lips for correcting a relief of this area, the injection being performed with the device as defined in claim 1, the method comprising:
performing an acquisition of the topography of the area, and
based on such acquisition of the topography, computing a volume of filler to inject into the skin, scalp, or lips via each microneedle.

17. The method of claim 16, wherein computing the volume of filler to inject depends on the localization of the microneedles on the area to treat when successive injections are carried out with displacement of the device relative to the area to be treated from one injection to the other, a succession of volumes of filler to inject being computed for respective injection sites of the area, each microneedle of the device configured to inject the same quantity of filler when injection takes place in one site, and this quantity varying when the device is positioned in another site.

18. The method of claim 16, wherein the quantity of filler to be locally injected is controlled as a function of the detected position of the device on the skin, scalp, or lips and of the correction to be applied.

19. A method of cosmetic treatment for correcting a relief of an area of skin, comprising delivering into the area a filler composition prepared by the method as defined in claim 16.

20. The method of claim 19, wherein the depth of injection of the microneedles into the skin is less than or equal to 500 micrometers.
